(19) United States
(12) Patent Application Publication
(10) Pub. No.: US 2004/0248874 A1
(43) Pub. Date: Dec. 9, 2004

(54) Anti-microbial composition comprising a metal ion chelating agent

(76) Inventors: Alan Edwin Jemmett, Angus (GB); Jack Schofield, Wirral Cheshire (GB); Russell Taylor, Ayrshire (GB)

Correspondence Address: ROPES & GRAY LLP, ONE INTERNATIONAL PLACE, BOSTON, MA, 02110-2624, US

(21) Appl. No.: 10/492,382

(22) PCT Filed: Oct. 14, 2002
(86) PCT No.: PCT/GB02/04662

(30) Foreign Application Priority Data
Oct. 12, 2001 (GB) 0124527.3
Aug. 2, 2002 (GB) 0217954.7

(51) Int. Cl. A61K 31/555

(52) U.S. Cl. 514/184

(57) ABSTRACT

The present invention relates to compositions suitable for use as topical pharmaceutical compositions for use in the treatment or prophylaxis of a superficial microbial species infection, and/or as anti-microbial cleansing compositions for use in the sanitary cleaning of animate or inanimate surfaces. The pharmaceutical compositions comprise a physiologically acceptable metal ion chelating agent and a pharmaceutically acceptable carrier therefor, in which composition said metal ion chelating agent has a metal ion chelating capacity for metal ions on which said microbial species is dependent for viability. The cleansing compositions comprise: a cleaning composition wherein is provided a metal ion chelating agent and in which composition said metal ion chelating agent has a metal ion chelating capacity for metal ions on which a microbial species is dependent for viability.

The present invention relates to anti-microbial compositions, and agents for use in preparing medicaments suitable for treating a microbial species infection(s).

It is a widely accepted and publicised fact that modern day medicine is facing a resurgence in bacterial infections and related diseases previously believed to have been treatable by administration of a variety of existing antibiotics on offer, but against which we now have a diminishing choice of effective therapies and medicaments. As our present day antibiotics are repeatedly used in an attempt to combat infections, microbial species are acquiring resistance to these pharmaceutical products due to genetic mutation.

One of the major drawbacks of existing antibiotics is their generally highly specific nature with a particular antibiotic being targeted to inactivate or attack a particular bacterial component, structure, enzyme or protein, for example, whose normal function is required for bacterial survival and/or replication. It is only necessary, therefore, for a bacterium to acquire an antibiotic-resistance-conferring mutation in the gene encoding the particular target of an antibiotic in order for the bacterium to circumvent attack by that antibiotic.

The situation is reaching the point where an increasing number of bacterial species are becoming resistant to particularly powerful antibiotics which were previously used only as a “last resort” treatment in cases where other therapies had been ineffective. Infectious agents such as methicillin resistant Staphylococcus aureus (MRSA) and vancomycin resistant species are proving a major problem. MRSA is spreading around the world and is responsible for many deaths. It is a particular problem in hospital environments with an increasing number of patients succumbing to hospital acquired MRSA infection during their hospital admission. When an infection breaks out in a hospital, theatres and wards need to be shut down or isolated, resulting in a reduction of available facilities within already overstretched health services and increased patient waiting times for treatment, in addition to the financial burden to replace bedding, mattresses etc for the previously infected hospital areas.

It is an object of the present invention to reduce or overcome one or more of the above disadvantages.

It has now been found that a chelating agent can be used to form a complex with metal ions utilised by metal ion dependent microbes, thereby effectively starving such microbes of a vital nutrient, and preventing their growth and proliferation and compromising viability.

Thus in a first aspect the present invention provides the use of a metal ion chelating agent for the manufacture of a medicament for the treatment or prophylaxis of a microbial species infection.

It is to be understood that the term a microbial species includes various species including bacterial species, mycobacterial species, fungal species, protozoal species and parasitic species. Typically the microbial species is a bacterial species, and may be a gram positive bacilli, a gram positive cocci, a gram negative bacilli or a gram negative cocci. A non-exhaustive list of bacterial species whose viability may be inhibited by the present invention includes Bacillus subtilis, Bacillus cereus, Bacillus anthracis, Corynebacterium species, Clostridium species, Staphylococcus aureus of the methicillin sensitive strain (for example Oxford strain) and MRSA strains (for example E15, E16, E16/79), coagulase negative Staphylococcus of methicillin sensitive and methicillin resistant strains, Streptococcus pyogenes, Streptococcus agalactiae, Streptococcus equisimlis, Enterococcus faecalis of vancomycin sensitive and vancomycin resistant strains, Enterococcus faecium of vancomycin sensitive and vancomycin resistant strains, viridans Streptococcus, Streptococcus pneumoniae, Escherichia coli, Shigella sonnei, Salmonella species, Klebsiella pneumoniae, Proteus mirabilis, Proteus vulgaris, Enterobacter cloacae, Vibro parahaemolyticus, Haemophilus influenzae, Legionella pneumophila, Pseudomonas aeruginosa, Pseudomonas fluorescens, Pseudomonas putida, Campylobacter species, Neisseria gonorhoeae, Moraxella catarrhulis, fusarium species. Particular fungal species whose viability may be inhibited by the present invention include Candida albicans, Candida glabaeata (torulopsis), Candida krusei, Candida tropicalis, Aspergillus niger.

While it will be appreciated that infection by the above microbial species cause pathology in human hosts, the present invention is also applicable to microbial species infections in other host species as well as humans, for example, livestock, and other domestic animals and wild animals. Examples of animal infections that may be overcome by administration of metal ion chelating agents include Trichomonas species infecetion, for example, Trichomonas vaginalis, and those causing digital dermatitis, udder dermatitis, horse mud fever.

The terms microbial species and microbe will be used inter changeably herein and are to be understood to refer to and mean the same, unless the context specifically requires otherwise.

It will be appreciated that for a microbial species to survive not only must its environment provide a source of requisite metal ions but they must be present in a bio-available form and at sufficient concentrations to meet the requirements of the microbial species. In the present invention the chelating agent will reduce the bio-available concentration of metal ions preferably to a level below a threshold level needed to support microbe survival. In some forms the metal ion chelating agent may undergo a chelation reaction having a relatively high equilibrium constant such that the chelating agent will chelate substantially all of a particular metal ion available, thereby prejudicing microbe viability. In other forms, for example, where a relatively high concentration or large amount of a particular metal ion is needed by a microbe, it may be possible to provide an effective medicament with a metal ion chelating agent that undergoes a chelation reaction having a relatively low equilibrium constant. While such a chelating agent may chelate a relatively small amount of the metal ion present, the level of bio-available metal ion may still be reduced sufficiently to reach a level below the threshold needed for microbe survival. The metal ion chelating agent can form a chelate with metal ions at the surface of a metal, in effect providing a barrier at the metal surface which prevents access of microbes to the metal. Accordingly it should be understood that references to “removal” of metal ions here include not only effectively total removal of bio-available metal ions for a microbial species in question but also a reduction in metal ion concentration to a level which prevents or substantially inhibits viability of the microbial species.

The processes within microbial species that are dependent on metal ions and that are required for microbial species viability are generally numerous and include processes of nutrition and reproduction such as DNA replication, cell division, protein synthesis, RNA synthesis. Particular metal ions required by microbial species which may be mentioned, include Zn²⁺, Mg²⁺, Mn2+, Co²⁺, Fe²⁺.

Preferred chelating agents can chelate various different metal ions and thereby attack microbes dependent on such different metal ions, by multiple routes. 8-hydroxyquinoline has been found to have a particularly broad spectrum of activity, chelating most metals apart from sodium, potassium and calcium. Other chelating agents which have a weaker chelating effect, may have an effective chelating activity with a narrower range of metals, but nevertheless can also be useful for treatment of a useful range of microbial species infections. This is particularly advantageous where a microbe can survive, though perhaps in a weakened state, by substituting a different metal ion to perform the functions of a particular metal ion that is chelated. Where a second different metal ion is also chelated and the microbial species is further weakened it is less likely that the microbial species will be able to proliferate successfully.

It will also be appreciated that not all microbial species will be dependent on the same metal ions for viability. By providing a metal ion chelating agent that removes a variety of metal ions, which may conveniently be referred to as the target metal ions, a single medicament can be provided that is effective against many microbial species that show dependence on different combinations, subgroups or individual metal ions from amongst the target metal ions. Thus, it is preferable for the metal ion chelating agent to form a chelate with a plurality of metal ions selected from Mg²⁺, Fe²⁺, Cu2+, Zn2+, Mn2+, Ni²⁺, and Se²⁺. Advantageously there is used a metal ion chelating agent which forms a chelate with at least one trace metal ion. The term trace metal ion is understood in the art to mean a metal ion whose presence is only required in minute amounts.

Typically the metal ion and the metal ion chelating agent form together a stable complex such that the metal ion is effectively removed for a sufficient period of time to prejudice microbe viability and overcome the infection before the metal ion chelate dissociates. In general the metal ion and metal ion chelating agent should form a stable chelate under physiological conditions.

It will be appreciated that there may be many different possible metal ion chelating agents that could effectively remove the metal ions of choice. It will, however, be appreciated that the metal ion chelating agent used should preferably exhibit low toxicity, and more preferably no toxicity, to the host organism to be treated. It will be understood, however, that the acceptable level of toxicity of the chelating agent will be assessed in relation to the severity of the microbial infection and to the administration route or mode of treatment. Where an infection has a high risk of resulting in mortality, severe disablement or severe symptoms a higher level of toxicity may be tolerated than in the case of a relatively mild and more inconsequential infection. Such considerations are well understood and routinely used in assessing therapeutic regimes in the field of the art.

Preferably the metal ion chelating agent is a heteropolar compound comprising at least one unsaturated heterocyclic six-membered ring in which at least one heteroatom moiety acts as a hydrogen acceptor and in which said compound also comprises at least one hydrogen donor moiety, conveniently a hydroxyl group, said heteropolar compound having no substituent which by itself or together with another substituent or substituents creates such steric hindrance and/or renders the molecule so basic or acidic or so alters the steric geometry of the molecule as to prevent interaction of the hydrogen donor and acceptor moieties of one molecule of heteropolar compound with the hydrogen donor and acceptor moieties of another molecule of said heteropolar compound

Whilst unsubstituted heteropolar molecules are preferred, substituents may be present on the heteropolar molecules, provided they do not singly or collectively prevent interaction of the hydrogen donor and acceptor moieties as by steric hindrance. Thus, for example, hydrocarbon substituents such as alkyl groups should not contain more than four carbon atoms, preferably not more than two carbon atoms. When the substituent is ortho to either the heteroatom or the hydroxyl group the steric hindrance effect is likely to be greater than when said substituent is in the meta or para position to either the heteroatom or a hydroxyl group. Alkene and alkyne substituents, carboxyl containing and amine containing substituents will all effect the activity of the heteropolar molecules and should be avoided.

In general the preferred metal ion chelating agent is a hetero aryl compound having at least one nitrogen in the ring structure and at least one hydroxyl substituent disposed on the ring structure so as to provide together, a chelating function. Preferred metal ion chelating agents are selected from optionally substituted 2,3-dihydroxypyridine; 4,6-dihydroxypryrimidine; 2-pteridinol; 2,4-quinolindiol; 2,3-dihydroxyquinoxalin; 2,4-pteridinediol; 6-purinol; 3-phenanthridinol; 2-phenanthrolinol; 2-phenazinilol, and most preferred is 8-hydroxyquinoline.

8-hydroxyquinoline has the advantage of forming metal ion chelates with a particularly broad range of different metal ions.

It will be appreciated that the route of administration of the metal ion chelating agent and the formulation thereof may vary depending on, for example, the microbial species in question, the patient or host organism, the site of infection, the severity of infection etc. It is preferable that the metal ion chelating agent be applied topically to a patient.

In a second aspect the present invention provides a topical pharmaceutical composition suitable for use in the treatment or prophylaxis of a superficial microbial species infection, said composition comprising a physiologically acceptable metal ion chelating agent and a pharmaceutically acceptable carrier therefor, in which composition said metal ion chelating agent has a metal ion chelating capacity for metal ions on which said microbial species is dependent for viability.

It is well understood in the field that pharmaceutical compositions are required to meet stringent safety requirements and those skilled in the art will be able to determine the types of carrier meeting these requirements and therefore being pharmaceutically acceptable.

The term physiologically acceptable metal ion chelating agent is to be understood to mean a metal ion chelating agent which has a metal ion chelating activity when administered to a patient which does not cause severe adverse effects to the physiological functioning of the patient's body. The degree of disruption of, or adverse effect to the normal physiology of the patient that may be tolerated may be assessed against the severity and symptoms resulting from the infection to be treated. Such considerations are well accepted and understood in the field.

The form of the composition of the present invention may include liquids, sprays, creams, ointments or pastes. Preferably the composition is a paste. These compositions can be readily applied topically and in the case of pastes, ointments or creams may be applied with relative ease to a particular region or restricted part of the body with minimal risk of the composition spreading to other parts of a body that are not to be treated. A paste is to be understood to be a generally thick substance with a degree of “sticking” or “setting” character, whereby the compositions can be maintained at the site of application for significantly longer than many other forms of composition, thus providing an ongoing treatment to the body part. The “sticky” or “setting” character can be achieved by inclusion of various components known in the art including polycellulose thickeners such as sodium carboxymethylcellulose, hydroxyethylcellulose, preferably hydroxyethylcellulose.

The polycellulose thickeners, such as hydroxyethylcellulose, have the additional advantage of controlling the pH of the composition. Maintaining the pH in the desired range is important as pH has been shown to affect the chelating activity of the metal ion chelating agent and, as described herebelow, also affects the flow of blood to the site of application.

It is further preferable to employ paste formulations which form a dry outer skin over the area to which it is applied, thereby effectively sealing off the area being treated. This helps to protect the area from the ingress of, for example, foreign matter and especially infectious agents, from the exterior. When a wound is being treated this is particularly advantageous.

It will be appreciated that the choice of components of the composition may be limited by the nature of the metal ion chelating agent. For example the preferred metal ion chelating agents 8-hydroxyquinoline; 2,3-dihydroxypyridine; 4,6-dihydroxypryrimidine; 2-pteridinol; 2,4-quinolindiol; 2,3-dihydroxyquinoxalin; 2,4-pteridinediol; 6-purinol; 3-phenanthridinol; 2-phenanthrolinol; 2-phenazinilol, are generally insoluble or only poorly soluble in aqueous solution. Suitable aqueous based compositions can be prepared by using an intermediate solvent such as a glycol, preferably monoethylene glycol or propylene glycol, and a wetting agent. Those skilled in the art will appreciate that a wide range of wetting agents are available that may be used which would give solubility of the metal ion chelating agent in glycol. Preferably the wetting agent is octylphenolethoxylate (commonly known as Synperionic OP10) or poly ethylene glycol tert-octyl phenyl ether (commonly known as Triton X-100).

It will be appreciated that a range of different proportions of the various components of the aqueous based compositions may be used depending on the solubilities of the metal ion chelating agents used, the final concentration required etc. In general we have found that the amount of wetting agent used is relatively sensitive. In the case of the intermediate solvent (glycol etc), once a required minimum amount sufficient for solubilisation of the metal ion chelating agent in the water is present, then the amount of this intermediate solvent can be readily increased further, though there is normally no particular advantage in doing so.

In the case of 8-hydroxyquinoline we have found that suitable proportions which may be used are typically:

| Component | Parts by weight |
|---|---|
| Metal ion chelating agent | 1 |
| Wetting agent | 4 ± 5% |
| Intermediate solvent (glycol) | at least 20 (preferably at |
| | least 40) |
| Water | as required to obtain |
| | desired final |
| | concentration of chelating |
| | agent |

In general the pharmaceutical compositions of the invention may be prepared by bringing the metal ion chelating agent into intimate admixture with a pharmaceutically acceptable carrier therefor. In the case of a chelating agent which is poorly soluble in water as noted above, the method generally comprises the steps of: mixing the metal ion chelating agent with a wetting agent and a non-aqueous water soluble solvent to produce a concentrate; and then diluting the concentrate with an aqueous diluent such as water, or water containing a thickener to provide a generally paste-form composition. Thus typically a metal ion chelating agent such as 8-hydroxyquinoline may be heated together with a wetting agent and glycol, preferably to at least 55° C. The cooled mixture can then be blended with a hydroxycellulose paste of water containing hydroxyethylcellulose. In this way the active ingredient can be dissolved in an aqueous diluent, such as the hydroxycellulose paste or other aqueous carrier. The glycol enables dispersion in an aqueous carrier, and the wetting agent enables dissolution of the chelating agent in the glycol.

As the metal ion chelating agents can be used at very low concentrations, it is generally convenient to utilise more or less concentrated compositions incorporating a water carrier (in order to reduce transportation and packaging costs etc) and then diluting these still further at the point of use.

Thus in general the composition can be further diluted in a hydroxycellulose paste, preferably to give a pharmaceutical composition paste containing the metal ion chelating agent at 1% to 0.01%, more preferably at 0.1% to 0.01%, further preferably at 0.05%, w/v. The concentration of hydroxycellulose in the hydroxycellulose paste can be chosen to provide a final pharmaceutical composition of a desired thickness and consistency. Where the pharmaceutical composition is a paste the concentration of hydroxycellulose in the composition is preferably at a level such that a flexible outer skin can form on the applied composition, without the underlying layers of the composition drying out completely.

The preferred paste compositions have a pH in the range from 7.5 to 10, most preferably 9.3 to 9.7. Such pharmaceutical composition paste compositions have the advantage of acting as a blood attractant thereby further promoting healing at the infected site. Such pastes can be likened to a “liquid bandage”, by setting on application to a greater or lesser extent, eliminating infection, aiding healing and protecting the site of application. Suitable water based carriers also encompass oil-in-water and water-in-oil, emulsions.

The metal ion chelating agent may also be presented in an oil-based carrier. Suitable oils include those with a high linoleic acid and linolenic acid content, more commonly known as omega 3 and omega 6 fatty acids, similar to fish oil or rapeseed oil. Preferably the oil carrier is selected from trout or salmon oil, more preferably salmon oil. Oil based formulations are typically applied by rubbing into the skin, and are useful where it is desired to deliver the metal ion chelating agent into lower dermal layer regions.

In other applications, for example where an infected area is to be washed or rinsed, or the presence of a more permanent set paste is undesirable, such as in the nasal passage, outer ear passage, vagina, mucosal surfaces etc it may be preferable to provide a liquid composition, conveniently one which can be applied as a spray. Suitable concentrations of the active metal ion chelating agent are generally 1 to 0.1%, more preferably at 0.1 to 0.01%, further preferably at 0.05%, w/v. Conveniently there may be used physiological saline as a carrier.

It will be appreciated that whilst the description hereinabove has described various aqueous based compositions, formulated in glycol, and oil based compositions, any suitable combination of the glycol and oil bases are included within the scope of the invention, and are of particular use when the present invention is in the form of a cream or lotion, for example.

In a third aspect the present invention provides an anti-microbial cleansing composition suitable for use in the sanitary cleaning of animate or inanimate surfaces, which composition comprises: a cleaning composition wherein is provided a metal ion chelating agent and in which composition said metal ion chelating agent has a metal ion chelating capacity for metal ions on which a microbial species is dependent for viability.

A cleansing composition according to the present invention is useful in a wide range of applications. A cleansing composition suitable for personal hygiene applications, such as a face or body wash, can be beneficial in the treatment and management of acne and related skin complaints. Such a wash could also be used in conjunction with a pharmaceutical composition according to the present invention as described hereinabove, in severe cases of the skin complaint. They may also be used to provide a general overall cleanliness where no skin problem is present.

Suitable body cleaning compositions for humans and/or animals to act as carrier for the metal ion chelating agent are well known in the art and their formulation can be readily ascertained. Such compositions would generally have the active metal ion chelating agent present at a concentration in the range 0.02 to 0.05%, w/v. It will be appreciated that the concentration will be dependent on the intended application of the cleansing composition i.e. whether it is to be further diluted as in, for example, a bath foam composition, or applied relatively undiluted or slightly diluted as in, for example, a face wash etc. The cleaning composition could also take the form of a bar of soap or the like.

Cleansing compositions of the invention suitable for use in the cleaning of inanimate surfaces such as kitchen appliances, kitchen surfaces, food preparation and/or storage areas and/or equipment, dishes, crockery, cutlery, glassware, bathroom appliances etc. where it is desirable to have an increased level of hygiene, generally include a detergent fluid in the carrier. In particular the use of such a kitchen cleansing composition and a body cleansing composition suitable for washing hands wash could be advantageous to reduce the risk of food poisoning resulting from microbial contamination when handling and preparing food.

While a general household cleaning composition for use in cleaning throughout the house, including the kitchen and bathroom, may be used as a suitable carrier for the metal ion chelating agent, it will be appreciated that where the cleansing composition is to be used in the kitchen or around food and accessories used with food, suitable cleaning compositions should generally include those known to be compatible with food preparation, in case these should not be adequately rinsed off after cleaning etc.

The anti-microbial cleansing composition could also be in the form of a laundering product for washing fabrics, for example, clothes, bed linen, operating theatre gowns, overalls etc.

An anti-microbial cleansing composition according to the present invention could also be used to clean appliances and systems such as air conditioning systems by spraying air passing through the system with a liquid cleansing composition, thereby cleansing the air that is to be vented to or from the system, of microbial species. Such an application of the present invention would be particularly beneficial in hospital air conditioning systems.

The cleansing composition could be used as part of the routine cleaning procedure to ensure an adequate level of hygiene and cleanliness is achieved in various environments where there is an increased requirement for sanitary conditions. Such a cleansing composition would also be of particular benefit in the disinfection of hospital, health centre, dental surgery, veterinary surgery and the like facilities and equipment used therein, including wards, operating theatres, beds, furniture and other inanimate objects that may have been contaminated with, or are at risk of contamination with, antibiotic-resistant strains of bacteria such as MRSA.

A cleansing composition could also be in the form of a hand wash. Such a hand wash would be of particular benefit to medical practitioners for use prior to examining or treating patients. Conveniently the cleansing composition could be in the form of a substantially non-aqueous self drying hand gel, thereby reducing the risk of transfer of microbes to the cleaned hands from towels etc. Alternatively the cleansing composition could be presented in the form of a wipe impregnated with a liquid cleaning composition of the invention. Cleaning wipes are widely used within hospitals, nurseries, around the household, as facial wipes in cleansing routines and for cleaning babies and young children etc. It will be appreciated that the metal ion chelating agent could be incorporated in a formulation of a suitable wipe at a level appropriate for the intended use of the wipe, for example, where a wipe is to be used to clean a wound or disinfect a body part prior to surgery etc a higher concentration of the metal ion chelating agent would be included than in a wipe such as a “freshening wipe” routinely provided for travellers during their journey on an air flight. Suitable concentrations in the latter applications would typically be in the range from 0.02 to 0.03%, w/v.

In a fourth aspect the present invention provides an article fabricated of natural or synthetic polymer for use in a medical application in which the presence of a microbial species is inimical, said article having a coating comprising a metal ion chelating agent, said coating of metal ion chelating agent having a metal ion chelating capacity for metal ions on which said microbial species is dependent for viability when said article is in use.

Such an article could take the form of apparel worn by medical practitioners, for example latex gloves, aprons or overalls formed of natural or synthetic polymer material such as rubber, polyethylene, polyvinyl chloride etc, or could take the form of a medical appliance such as catheters, gastro-nasal tubes, laparoscopic instruments etc.

Articles according to the present invention could also be of general use for patients with a compromised immune system, such as transplantation patients undergoing or who have undergone organ, bone marrow or the like transplantation.

The coating could be simply applied by dipping the article in a liquid composition of chelating agent such as a liquid pharmaceutical composition described herein. Alternatively the coating could take the form of a powder sprinkled or dusted onto the article.

In a fifth aspect the present invention provides a method of treatment or prophylaxis of a microbial species infection comprising the administration, to a human or animal in need of such treatment, of an effective dose of a metal ion chelating agent, said metal ion chelating agent having a metal ion chelating capacity for metal ions on which said microbial species is dependent for viability.

The metal ion chelating agent could be administered to the patient, for example, by washing, spraying or bathing an infected area with a liquid pharmaceutical composition of the invention; by applying a cream, ointment, paste etc pharmaceutical composition of the invention to an infected area, or by any other suitable route of administration that would result in removal of metal ions required by the microbial species for survival.

Thus, for example, skin infections such as acne or infected wounds could be washed or bathed with the metal ion chelating agent compositions of the invention; body passages, such as the vagina or nasal passage, could be sprayed to treat infections such as vaginal Trichomonas infection; bovine digital dermatitis could be treated by application of a paste of the metal ion chelating agent to the animals affected hoof; creams containing the metal ion chelating agent may be applied to sores on skin, etc. It will be appreciated that these examples merely serve to illustrate a few of the ways in which different microbial infections may be treated and the metal ion chelating agent administered. The physician in question administering the treatment will be capable of determining the most appropriate route of administration on a case by case basis where necessary.

Further preferred features and advantages of the invention will appear from the following detailed examples provided by way of illustration.

### EXAMPLE 1

### Preparation of Metal Ion Chelating Agent Glycol Based Concentrate for Use in Preparation of Pharmaceutical Composition and Anti-Microbial Cleansing Composition

10 g of 8-hydroxyquinoline was dissolved at 55° C. in 40 g of octylphenolethoxylate (Synperionic OP10) or poly ethylene glycol tert-octyl phenyl ether (Triton X-100) with 200 g of propylene glycol or monoethylene glycol. The mixture was cooled to room temperature and blended with further glycol to a total weight of 500 g to give a concentration of 2% 8-hydroxyquinoline.

### EXAMPLE 2

### Preparation of a Topical Paste Pharmaceutical Composition

One part of the glycol based concentrate prepared according to Example 1 described hereinabove was mixed with 39 parts of a paste of de-ionised water containing 6% hydroxyethylcellulose (by weight) (although somewhat more e.g. up to 9% could also be used) to give a paste composition containing 0.05% w/v of 8-hydroxyquinoline. The pH of the composition was adjusted, if necessary, to 9.3-9.5 by adding a small amount of NaOH as required.

### EXAMPLE 3

### Preparation of a Liquid Pharmaceutical Composition

One part of the glycol based concentrate prepared according to Example 1 was diluted in 40 parts de-ionised water containing 5% hydroxyethylcellulose (by weight). The pH of the composition was adjusted, if necessary, to 9.3-9.5 as described above. The liquid pharmaceutical composition was suitable for use as a spray.

### EXAMPLE 4

### Preparation of Metal Ion Chelating Agent Oil Base Concentrate

20 g of 8-hydroxyquinoline was dissolved in 980 g of salmon oil. The pH of the composition was adjusted, if necessary, to 9.3-9.5 as described above. This oil-based concentrate was suitable for further dilution in oil bases suitable for pharmaceutical compositions or cleansing compositions.

### EXAMPLE 5

### Preparation of an Anti-Microbial Cleansing Composition

One part of the aqueous based concentrate prepared according to Example 3 was further diluted in water to be used at 1 in 20 parts. The hydroxyethylcellulose content gives a lathering effect.

### EXAMPLE 6

### Treatment of Acne

A 26 year old male who had suffered with acne vulgaris affecting the skin on the back from the age of about 16 years was treated with a twice daily application of the paste composition of Example 2 for two weeks. Within the first 3 to 4 days the generalised erythema surrounding the pustules appeared to clear. This was followed by a period where the condition appeared to be quiescent, but static with no new pustules appearing. Towards the end of the second week of treatment there was dramatic improvement in the condition with almost complete disappearance of the papules and pustules. The subject stated that he had never felt the skin on his back in the affected area to be so smooth. There were no untoward side effects experienced or reported.

### EXAMPLE 7

### Treatment of Sinus Infection

A female subject who had suffered a long term sinus infection for some 14 to 15 years which had proved resistant to treatment with a wide range of antibiotics (including Trimox, Veetids, Cipro, Doxycycline and Clindramycin), was treated for 10 days with twice daily applications of the paste composition of Example 2 inside the nostrils using cotton buds. The symptoms had substantially disappeared after 7 days and did not reappear after completion of the treatment.

### EXAMPLE 8

### Treatment of Digital Dermatitis

Five freshly calved heifers with mild-moderate digital dermatitis were treated with daily or bi-daily applications of the paste composition of Example 2, in place of the normally used oxytetracycline spray. All cases healed at the end of approximately 1 week.

### EXAMPLE 9

### In vitro Testing

The concentrate of Example 1 and liquid composition of Example 3 were tested against the micro-organisms listed below. The micro-organisms were obtained from clinical specimens. Wells were cut into appropriate agar plates (nutrient, DST lysed, isosensitest agar) and filled with 15 μl of the concentrate or liquid composition. The micro-organisms were innoculated onto the agar and lawn plated to give a semi-confluent growth. Plates were incubated for 24 hours at 37° C. under appropriate atmospheric conditions, after which the growth inhibition zone sizes were noted.

Micro-Organisms Tested:

Bacillus subtilis, Bacillus cereus, Corynebacterium species, Staphylococcus aureus (Oxford strain—methicillin sensitive strain; E15, E16, E16/79—MRSA strains), coagulase negative Staphylococcus (methicillin sensitive and methicillin resistant strains), Streptococcus pyogenes, Streptococcus agalactiae, Streptococcus equisimlis, Enterococcus faecalis (vancomycin sensitive and vancomycin resistant strains), Enterococcus faecium (vancomycin sensitive and vancomycin resistant strains), viridans Streptococcus, Streptococcus pneumoniae (including intermediate penicillin resistant strains), Escherichia coli, Shigella sonnei, Salmonella species, Klebsiella pneumoniae, Proteus mirabilis, Proteus vulgaris, Enterobacter cloacae, Vibro parahaemolyticus, Haemophilus influenzae, Pseudomonas aeruginosa, Neisseria gonorhoeae, Moraxella catarrhulis, Candida albicans, Candida glabaeata (torulopsis), Candida krusei, Candida tropicalis.

Results:

Medium to very large zones of growth inhibition were observed for all the above micro-organisms with the exception of Pseudomonas aeruginosa (only small zone of inhibition obtained when using the concentrate).

1. A topical pharmaceutical composition suitable for use in the treatment or prophylaxis of a superficial microbial species infection, said composition comprising a physiologically acceptable metal ion chelating agent and a pharmaceutically acceptable carrier therefor, in which composition said metal ion chelating agent has a metal ion chelating capacity for metal ions on which said microbial species is dependent for viability.

2. An anti-microbial cleansing composition suitable for use in the sanitary cleaning of animate or inanimate surfaces, which composition comprises: a cleaning composition wherein is provided a metal ion chelating agent and in which composition said metal ion chelating agent has a metal ion chelating capacity for metal ions on which a microbial species is dependent for viability.

3. A composition according to claim 1 or claim 2 wherein said metal ion chelating agent is a heteropolar compound comprising at least one unsaturated heterocyclic six-membered ring in which at least one heteroatom moiety acts as a hydrogen acceptor and in which said compound also comprises at least one hydrogen donor moiety, said heteropolar compound having no substituent which by itself or together with another substituent or substituents creates such steric hindrance and/or renders the molecule so basic or acidic or so alters the steric geometry of the molecule as to prevent interaction of the hydrogen donor and acceptor moieties of one molecule of heteropolar compound with the hydrogen donor and acceptor moieties of another molecule of said heteropolar compound.

4. A composition according to any one of claims 1 to 3 wherein said metal ion chelating agent is a hetero aryl compound having at least one nitrogen in the ring structure and at least one hydroxyl substituent disposed on the ring structure so as to provide together, a chelating function.

5. A composition according to claim 4 wherein said metal ion chelating agent is selected from optionally substituted 2,3-dihydroxypyridine; 4,6-dihydroxypryrimidine; 2-pteridinol; 2,4-quinolindiol; 2,3-dihydroxyquinoxalin; 2,4-pteridinediol; 6-purinol; 3-phenanthridinol; 2-phenanthrolinol; 2-phenazinilol, and 8-hydroxyquinoline.

6. A composition according to claim 5 wherein said metal ion chelating agent is 8-hydroxyquinoline.

7. A composition according to any one of claims 1 to 6 wherein said metal ion chelating agent is a metal ion chelating agent which can form a chelate with any one of at least two different metal ions.

8. A composition according to any one of claims 1 to 7 wherein said metal ion chelating agent is a metal ion chelating agent which can form a chelate with at least one trace metal ion.

9. A composition according to any of claims 1 to 8 wherein said metal ion chelating agent is a metal ion chelating agent which can form a stable metal chelate under physiological conditions.

10. A composition according to any one of claims 1 to 9 which includes a wetting agent.

11. A composition according to claim 10 wherein the wetting agent is selected from octylphenolethoxylate and poly ethylene glycol tert-octyl phenyl ether.

12. A composition according to any one of claims 1 to 11 wherein said composition contains an intermediate solvent in the form of a non-aqueous water soluble solvent.

13. A composition according to claim 12 wherein said intermediate solvent is a glycol.

14. A composition according to claim 13 wherein said intermediate solvent is selected from monoethylene glycol and propylene glycol.

15. A composition according to any one of claims 1 to 14 wherein is included a thickener.

16. A composition according to claim 15 wherein said thickener is a polycellulose thickener.

17. A composition according to claim 16 wherein said thickener is hydroxyethylcellulose.

18. A composition according to any one of claims 1 to 17 comprising 1 part by weight of 8-hydroxyquinoline, 4±5% parts by weight of wetting agent, at least 20 parts by weight of glycol, and water.

19. A composition according to any one of claims 1 to 17 wherein said composition is in the form of a liquid, spray, cream, ointment or paste.

20. A pharmaceutical composition according to claim 1 or any one of claims 3 to 19 when dependent on claim 1 in which said metal ion chelating agent is present at a concentration of from 1% to 0.01% w/v.

21. A composition according to claim 20 in which said metal ion chelating agent is present at a concentration of from 0.5% to 0.05% w/v.

22. A pharmaceutical composition according to claim 1 or any one of claims 3 to 21 when dependent on claim 1, which composition has a pH in the range from 7.5 to 10.

23. A pharmaceutical composition according to claim 22 which composition has a pH in the range from 9.3 to 9.7.

24. A composition according to any one of claims 1 to 23 which includes a buffer to control the pH of said composition.

25. A composition according to any one of claims 1, 3 to 9 or 19 to 24 in which said metal ion chelating agent is provided in an oil-based carrier.

26. A composition according to claim 25 wherein said oil-based carrier is selected from trout oil, salmon oil and rapeseed oil.

27. A cleansing composition according to claim 2 or any one of claims 3 to 19 when dependent on claim 2, in which said metal ion chelating agent is present at a concentration of from 0.1 to 0.001% w/v.

28. A method of preparing a composition according to claim 1 or claim 2 comprising the steps of bringing the metal ion chelating agent into intimate admixture with a pharmaceutically acceptable carrier therefor.

29. A method according to claim 28 which method comprises the steps of mixing the metal ion chelating agent with a wetting agent and a non-aqueous water soluble solvent to produce a concentrate; and then diluting the concentrate with an aqueous diluent.

30. A method of preparing a paste composition according to claim 29, wherein said aqueous diluent is an aqueous thickener.

31. A method according to claim 30 wherein said metal ion chelating agent is 8-hydroxyquinoline and said 8-hydroxyquinoline is heated together with the wetting agent and glycol to at least 55° C.; and the mixture is blended with a hydroxycellulose paste of water containing hydroxyethylcellulose.

32. A cleansing composition according to claim 2, claim 27, or any one of claims 3 to 19 when dependent on claim 2, wherein said cleansing composition is in the form of a self drying hand gel.

33. The use of a metal ion chelating agent for the manufacture of a medicament for the treatment or prophylaxis of a microbial species infection.

34. An article fabricated of natural or synthetic polymer for use in a medical application in which the presence of a microbial species is inimical, said article having a coating comprising a metal ion chelating agent, said coating of metal ion chelating agent having a metal ion chelating capacity for metal ions on which said microbial species is dependent for viability when said article is in use.

35. A method of treatment or prophylaxis of a microbial species infection comprising the administration, to a human or animal in need of such treatment, of an effective dose of a metal ion chelating agent, said metal ion chelating agent having a metal ion chelating capacity for metal ions on which said microbial species is dependent for viability.
